(19) United States
(12) Patent Application Publication
(10) Pub. No.: US 2023/0018121 A1
(43) Pub. Date: Jan. 19, 2023

(54) USE OF AGAVE EXTRACTS FOR PROTECTING CAPILLARY FIBRE

(71) Applicant: LABORATOIRES DE BIOLOGIE VEGETALE YVES ROCHER, LA GACILLY (FR)

(72) Inventors: Sylvie TUFEU, PARIS (FR); Julie ONGENAED, ISSY-LES-MOULINEAUX (FR); Sevag TCHALEKIAN, ISSY-LES-MOULINEAUX (FR)

(21) Appl. No.: 16/492,598

(22) PCT Filed: Mar. 6, 2018
(86) PCT No.: PCT/FR2018/050505

§ 371 Date: Nov. 29, 2019

(30) Foreign Application Priority Data
Mar. 7, 2017 (FR) 1751833

(51) Int. Cl. A61K 8/9794; A61K 8/72; A61K 8/73; A61K 8/04; A61K 8/06; A61K 8/02; A61Q 5/00

(52) U.S. Cl. CPC A61K 8/9794; A61K 8/72; A61K 8/73; A61K 8/042; A61K 8/06; A61K 8/022; A61Q 5/002

(57) ABSTRACT

The invention relates to a non-therapeutic cosmetic use of fructans of Agave tequilana, containing natural inulins for protecting capillary fibre by improving the hydrophobicity of the capillary fibre. The invention also relates to a non-therapeutic cosmetic method comprising the therapeutic application of fructans of Agave tequilana containing inulins having a degree of polymerisation greater than 10, in order to protect the capillary fibre by improving the hydrophobicity of the capillary fibre, said application being optionally followed by rinsing.

### DESCRIPTION

### Technical field

The present invention relates to the non-therapeutic cosmetic use of fructans for protecting the hair fiber by improving the hydrophobicity of the hair fiber.

The present invention has its application in the field of cosmetics, more particularly of hair cosmetics.

In the description below, the references between square brackets ([ ]) refer to the list of references presented at the end of the text.

### Prior art

Like the skin, hair accumulates damage caused by environmental stress such as UV radiation, pollution, cold, but also esthetic treatments such as drying, curling, straightening, bleaching or dyeing, or the use of aggressive products during too frequent or unsuitable shampooing operations. Moreover, they are affected by internal factors such as stress, hormonal modifications or else the type of food.
In its visible portion, the hair is formed of 3 layers:
the cuticle, from 3.5 to 4.5 μm in thickness, is the outermost layer. It consists of horny plates in the form of scales which partially overlap one another and constitutes an impermeable protection for the hair shaft,
next is the cortex, representing 80% of the hair. It is composed mainly of proteins (essentially keratin) and gives the fiber its strength and solidity through its high level of organization. The cortex contains melanin granules that give the hair its color,
finally, the medulla, composed of dead cells, which has a soft, greasy texture.

The outer layer of the cuticle, and hence of the surface of the hair, consists of fatty acids. These bonded and unbonded fatty acids present at the surface of the hair act as a protective layer making the surface of the hair hydrophobic. The hydrophobicity of the hair is an important parameter for keeping it healthy. In principle, this property makes it possible to avoid too long-lasting a presence of water which promotes the growth of bacteria. Moreover, this barrier makes it possible to limit and/or prevent the penetration of other compounds present in the external environment. Hair in a good state may nonetheless absorb more than 30% of its own weight in water. Its length may then increase by 2% and its diameter by 15% to 20%.

Repeated attacks deteriorate this lipid film. When the cuticle is damaged, the hair becomes too porous, and the water penetration phenomenon is amplified, with the hair then being able to absorb 45% of its own weight in water. This massive absorption of water destabilizes the interactions between the proteins and thus causes it to lose strength and tenacity.

A damaged cuticle thus corresponds to a hair which is too hydrophilic, originating therefore from a deterioration of its cuticle which no longer performs its natural function of barrier before the cortex. The more the cuticle of the hair shaft is altered, or even destroyed, the more the keratin of the cortex absorbs water and loses its strength and elasticity.

Among the cosmetic raw materials used for hair care are conditioners which have the aim of making the hair easier to style, softer and shinier.

These conditioners may be cationic surfactants or silicone derivatives.

There are also inulins modified by chemical grafting of a group of hydrophobic nature to the base molecular structure of the inulin (W0200790554 ([1])). This chemical group may be of different forms and frequently corresponds to a hydrophobic carbon-based chain. This modification confers conditioning properties on these inulins. These inulins commonly originate from the plant source most conventionally used for their extraction: chicory.

Nonetheless, there remains a real need to find novel compositions and/or compounds of plant and/or natural origin that make it possible to effectively improve the protective properties of damaged hair, and in particular the hydrophobicity properties.

### Description of the Invention

The aim of the present invention is specifically to meet these requirements and respond to these drawbacks of the prior art.

The inventors are the very first to use agave extracts, making it possible to specifically effectively meet the abovementioned requirements.

The Applicant has identified, surprisingly, that some fructans, and especially some natural inulins of Agave tequilana, that is to say which are not chemically modified, have a hydrophobic action enabling protection of the hair fiber, despite their hydrophilic nature.

Indeed, the Applicant demonstrated, after significant amounts of research, that the use of some inulins makes it possible to significantly improve the hydrophobicity of the hair fiber, especially of damaged hair.

Surprisingly, the inulins used in the context of the invention, despite their hydrophilic nature, enable an improvement in the hydrophobic properties of the hair. Thus, a first subject of the invention relates to the non-therapeutic cosmetic use of fructans of Agave tequilana comprising natural inulins for protecting the hair fiber by improving the hydrophobicity of the hair fiber.

Agave is a monocotyledonous plant currently classified in the family of the Agavaceae. The most common species are Agave tequilana, Agave americana and Agave attenuata. Agave is endemic on the American continent and is present from the south of Canada to the north of South America and the Caribbean Islands, in particular in Mexico and throughout Central America (María de la Soledad Alonso Gutiérrez, thesis: “Valorisation de la bagasse de l'Agave tequilana W. cv azul: caractérisation, étude de la digestibilité et de la fermentation des sucres”[Utilizing bagasse of Agave tequilana W. cv azul: characterization, study of digestibility and of sugar fermentation], 2005 ([2])).

Agaves have an underground part having the form of a rhizome, and an aerial part having long stems and large fibrous leaves, arranged in the form of a rosette and tipped with a thorn. The flowers are located at the end of the stem and are greeny yellow in color (ALONSO GUTIÉRREZ ([2])). Inulin is a storage polysaccharide found in plants of the family Asteraceae, especially in the bulbs and roots of dahlias, Jerusalem artichokes, artichokes, dandelions and chicory, the latter being the source most widely used industrially. Inulin consists of 30 to 40fructose units connected by β (1-2) glycosidic linkages and of one or two glucose molecules placed at the chain end, and is in reality a glucofructosan. Agave inulins are carbohydrate reserves of fructan type (also referred to as fructosans). Polyfructosans were extracted from 8 year old Agave tequilana Weber var. azul. The result of the characterization of these fructosans gave a degree of polymerization of between 3 and 29, and a complex composition of fructooligosaccharides with β (1-2) and β (2-6) linkages, different from the inulins encountered in other plants (López, M.G. ; Mancilla, N.A. ; Mendoza-Diaz, G. (2003). : Molecular structures of fructans from Agave tequilana Weber var. azul. J. Agric. Chem. 51, 7835-7840 ([3]) ; ALONSO GUTIÉRREZ ([2])). Chicory inulins have a different structure from that of agave inulins. Indeed, chicory inulins have linear chains of fructoses connected by beta 2-1 linkages whereas agave inulins, even though they are also mainly composed of fructose, have lateral structures branched from the main structure by beta 2-6 linkages (López, M.G. et al. ([3])).

For the purposes of the present invention, “fructans of Agave tequilana” is intended to mean fructans originating from an extract of the plant. The fructans may be extracted by any process known to those skilled in the art, for example by extraction of the carbohydrates from the bagasse, clarification of the crude liquor obtained at the end of extraction then separation to obtain a fraction of fructans. The fructans of Agave tequilana generally have a degree of polymerization ranging from 3 to 60, more particularly from 3 to 29.

More particularly, the use of the invention is understood for natural inulins.

For the purposes of the present invention, “natural inulins” are intended to mean fructans of Agave tequilana that are not chemically modified, in particular that do not comprise any chemically grafted groups with a hydrophobic nature. These natural inulins therefore have a hydrophilic nature, which distinguishes them doubly from chemically modified chicory inulins used as conditioners, namely by their polysaccharide structure and their physicochemical behavior in relation to water. They have a degree of polymerization greater than 10, possibly ranging for example from 10 to 60, or from 10 to 29. The inulins may be obtained by any process known to those skilled in the art, for example that described by ALONSO GUTIÉRREZ ([2]). An example of natural inulin may for example be the product Metlin® (NEKUTLI), characterized by true inulins having a degree of polymerization greater than 10.

For the purposes of the present invention, “protecting the hair fiber” is intended to mean the act of entirely or partially repairing the damage caused by environmental stress, for example UV radiation, esthetic treatments such as drying, curling, straightening and dyeing, or the use of aggressive products. Advantageously, the effect of the fructans, and especially of the inulins used in the context of the invention, enables the improvement of the hydrophobicity of the hair fiber.

For the purposes of the present invention, “hydrophobicity of the hair fiber” is intended to mean the hydrophobic nature of the surface of the hair, associated with the presence of fatty acids forming the outer layer of the cuticle.

Surprisingly, the effect of improving the hydrophobicity of the hair fiber is not associated with the hydrophobic properties of the inulin itself, since the inulins used in the invention have a hydrophilic nature. Thus, the fructans and especially the inulins described in the invention make it possible to nourish the hair.

Advantageously, the improvement in the hydrophobicity is at least 6%, for example between 6 and 14% depending on the ratios, relative to the hydrophobicity measured before using the inulins according to the invention. This improvement may be measured using a tensiometric test, for instance the Wilhelmy test on hair fibers (SCHULZE ZUR WIESCHE et al.: “Prevention of hair surface aging”, J. Cosmet. Sci., 62, 237-249 (March/April 2011) Henkel AG & Co. KGaA, Hohenzollemring 127-129, 22763 Hamburg, Germany (E.S.z.W.), DWI an der RWTH Aachen e.V., Pauwelsstr. 8, 52056 Aachen, Germany (A.K., K.S.), and School of Materials, The University of Manchester, Manchester M13 9PL, UK (F-J.W.) ([4])).

In the context of the use of the invention, the fructans may further comprise fructooligosaccharides having a degree of polymerization less than or equal to 10. Advantageously, the fructooligosaccharides having a degree of polymerization less than or equal to 10 have an effect of improving the growth of the hair and/or the appearance of the hair and/or of the scalp. It may for example be a degree of polymerization ranging from 3 to 10, for example from 3 to 9, or from 4 to 8. It may for example be the commercial product Metlos® (NEKUTLI), characterized by fructooligosaccharides having a degree of polymerization less than or equal to 10.

In the context of the invention, the ratio of inulins to fructooligosaccharides used may be from 100/0 to 0/100, the limit value of 0/100 optionally not being included. The ratio may for example be 75/25, or 25/75, or 50/50.

Advantageously, the fructans are present in a cosmetic composition comprising a cosmetically acceptable support. The cosmetically acceptable support may for example be chosen from the group comprising shampoos, lotions, especially hair lotions, creams, mousses, milks, sprays, suspensions and serums.

The cosmetic composition advantageously has all the technical effects afforded by the extract of Agave tequilana described above, and may further comprise an acceptable carrier and from 0.05 to 5% by weight of agave extracts relative to the total weight of the composition, said agave extract comprising inulins having a degree of polymerization of from 10 to 60 and optionally fructooligosaccharides having a degree of polymerization less than or equal to 10. All the characteristics of agave extracts mentioned above for the use of the invention, and also all the characteristics of the use of an extract described above, apply mutatis mutandis to the 2 5 composition of the invention.

Advantageously, the cosmetic composition may comprise a mixture of fructans of Agave tequilana consisting (i) of inulins having a degree of polymerization greater than 10 and (ii) of fructooligosaccharides having a degree of polymerization less than or equal to 10, wherein the ratio of inulins to fructooligosaccharides is equal to 50/50.

In the present invention, “cosmetic composition” is intended to mean any composition intended for cosmetic, that is to say esthetic, use, a composition that may be brought into contact with the superficial parts of the human body, for example the epidermis and the body hair and head hair systems. Advantageously, a cosmetic composition exclusively or primarily makes it possible to protect them, fragrance them, keep them in a good state, modify their appearance or correct their superficial esthetic defects. The composition of the invention may especially contain a cosmetically acceptable carrier.

“Cosmetically acceptable carrier” is intended to mean a carrier suitable for use in contact with human and animal skin cells, in particular the cells of the epidermis, without toxicity, irritation, induced allergic reaction and the like, and adjusted to a reasonable advantage/risk ratio.

The cosmetically acceptable carrier may for example be chosen from water, glycerol, aloe vera gel, an aqueous plant extract, surfactants of natural origin, this list being nonlimiting.

The composition of the invention may be obtained by any suitable process known to those skilled in the art for the production of a cosmetic composition. This may for example be simple mixing. It may alternatively for example be a process comprising a step of incorporating an internal phase into an external phase by means of an emulsifier, for example a turbomixer of rotor-stator type. It may also be, for example, a process using the Phase Inversion Temperature (PIT) process, this process conventionally being used by those skilled in the art to obtain oil- in-water emulsions in which the dispersed droplets are particularly fine, for example with a diameter of from 0.1 to 1 μm.

The cosmetic composition may comprise from 0.05 to 5% by weight of agave extracts relative to the total weight of the composition, for example from 0.1 to 5%, from 0.1 to 1%, from 0.25 to 1% or from 0.25 to 0.5%.

The cosmetic composition of the present invention may be in any form suitable for a cosmetic application. Advantageously, the composition is a composition for topical use which may be rinsed out or left on.

It may for example be a composition in a form chosen from the group comprising an oil- in-water or water-in-oil emulsion or a mixture of these emulsions.

According to the invention, the cosmetic composition may for example be in a form chosen from the group comprising an aqueous or aqueous-alcoholic gel, an aqueous or aqueous- alcoholic cream and an aqueous or aqueous-alcoholic lotion. These formulations of use for the implementation of the present invention are known from the prior art by formulators. In these composition examples, it is sufficient to add agave extracts of the present invention in order to obtain a composition in accordance with the present invention.

According to the invention, the composition may be in a form chosen from a shampoo, a salve, a cream, an oil, a milk, an ointment, a powder, a solution, a gel, a serum, a balm, a butter, a lotion, a suspension, a soap, a spray or an emulsion, this form possibly being rinsed out or left on.

The extract of the present invention may be used in a cosmetic composition alone or in combination with other cosmetically or dermatologically active or inactive substances or ingredients. The inactive substances or ingredients are those which do not act cosmetically or dermatologically. These are elements of the composition which make it possible in particular to accompany the extract, to form a particular formulation, to keep the extract active over time, this list being nonlimiting. In other words, they may be any base product which may be found in conventional cosmetic or dermatological compositions. In opposition thereto, the active substances or ingredients are those which, in the targeted cosmetic or dermatological application, have an esthetic and/or medical action.

Thus, the agave extract of the present invention may be the sole active substance or ingredient of a composition or it may be combined with other active substances or ingredients of a cosmetic or dermatological composition.

A device is also described within the context of the invention, possibly being in a form chosen from a pot, a bottle, a pump bottle, a tube, a welded sachet, a mask, a spray, said device comprising an extract or a composition according to the invention.

Another subject of the invention relates to a non-therapeutic cosmetic process comprising the therapeutic application of fructans of Agave tequilana comprising inulins having a degree of polymerization greater than 10, for protecting the hair fiber, said application being optionally followed by rinsing.

In the context of the cosmetic processes according to the invention, or of the use according to the invention, the use is understood as a non-therapeutic use, for example for treating healthy skin and hair, that is to say in particular scalps that do not exhibit a diseased state or, if the scalp does exhibit a diseased state, for a strictly esthetic use, excluding any therapeutic use. Indeed, the cosmetic and esthetic use of the invention is not associated with an inevitable therapeutic effect.

Thus, any cosmetic use and any cosmetic process according to the invention are, respectively, non-therapeutic cosmetic uses and non-therapeutic cosmetic processes that do not aim to treat a disease.

In a particular embodiment, the application is not followed by rinsing.

Regardless of the embodiment, the application may be carried out on natural hair, damaged hair, dry hair, dyed hair or brittle hair.

Other advantages may also become apparent to those skilled in the art on reading the examples below, illustrated by the appended figures and given by way of illustration.

### EXAMPLES

### Example 1: Preparation of the Agave Extracts

The agave extracts may be the commercial products Metlos® (fructooligosaccharides with a degree of polymerization less than or equal to 10), and/or Metlin® (inulins with a degree of polymerization greater than 10), supplied by Nekutli. The raw material used for the production of Metlos® and Metlin® is organic blue agave harvested in the high plateaus of the state of Jalisco.

After 5 to 7 years of carefully controlled growth, the agave has the maximum amount of fructans and is therefore ready to be converted.

The agave is received fresh and washed on arrival to eliminate as much foreign matter as possible. The agave is then ground in three steps to go from 40 to 100 kg of agave core to a pulpy bagasse, ready to be converted in order to extract the carbohydrates therefrom.

The crude liquor obtained from the extraction is subsequently clarified in order to eliminate any remaining color and impurities. The clarified juice is subsequently purified into two fractions: Metlos® and Metlin®. Each product is subsequently taken to a last purification step in order to eliminate all the minerals to obtain a high level of purity (>99% carbohydrates).

### Example 2: Evaluation of the Effect of the Inulins by Tensiometric Measurements of the Surface of the Hair

The aim of this study is to evaluate the effect of Metlin® and Metlos®, denoted respectively active agent A and active agent B, by measurements of the water contact angle on the surface of the hair.

Healthy hair is naturally protected by a surface lipid layer with hydrophobic properties. However, the hydrophobicity of hair decreases after chemical damage, caused for example by bleaching. The aim of the present study is to measure the increase in the hydrophobicity on the surface of the hair after immersion in a solution of nourishing active agents based on fructans (inulins and/or fructooligosaccharides), due to the improvement in the hydrophobicity of the hair.

Principle of the study

The measurement is based on the Wilhelmy method. This is a method currently used to measure the contact angle of a liquid on a single fiber. The fiber (the hair) must not be permeable to the liquid used (water) and the surface tension of this liquid must be known.

The principle consists in using high-precision scales in order to measure the force required during the immersion of the hair in the water. The contact angle is calculated from the Wilhelmy equation:

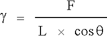

with:
γ: surface tension of water: 72.8 mN/m
F: force
L: length in the wet state (measured beforehand)
θ: contact angle

As indicated above, the measurement of the contact angle requires prior determination of the wetted length of the sample. The measurement of the wetted length is carried out using a solvent with a low surface tension, hexane, which has a surface tension of 18.4 mN/m, the contact angle of which is considered to be zero.

Regarding the measurement of the contact angle, the sample is gradually immersed in hexane, and the immersion force is measured. The force F exerted just in contact with the solvent is determined by linear regression, there and by calculation, and the wetted length is determined with the Wilhelmy equation.

Test data

| | |
|---|---|
| Lock | Natural—treated |
| T Nat | Natural standard (European brown, supplier: Quimdis) |
| D2 | D2—Standard |
| D2 + | D2 + 1 application by immersion, rinsed or not rinsed in |
| treatment | the active agent solution |

Origin of the Hair: Brown European Hair.

The hair denoted “D2” or “D2 standard” is brown European hair (supplier: Quimdis) that has undergone 2 cycles of bleaching. Bleaching is the lightening of the hair's natural shade by chemical modification (alkaline oxidation processes) of the features of the melanin pigments. The highly gradual process of bleaching the pigment (solubilization by depolymerization of melanin) is preceded by an attack on the keratin and the polypeptides stabilizing the melanin pigment by the bleaching treatment (breaking of disulfide, amide, salt and hydrogen bonds). Hair that has undergone bleaching repeated twice is marked D2. We carry out this process in the laboratory by immersing the locks in a solution based on sodium persulfate and hydrogen peroxide.

The inulins or fructooligosaccharides used are Metlin® or Metlos® (Nekutli) and mixtures thereof, the Metlin®/Metlos® ratios of which are as follows: 100/0, 75/25, 50/50, 25/75 and 0/100.

The concentrations of the inulins and/or fructooligosaccharides in the aqueous solutions of active agents applied are 0.25%, 0.5% and 1%. This percentage corresponds to the total product in the product, that is to say either inulin, when the product does not contain fructooligosaccharides, or fructooligosaccharides when the product does not contain inulin, or the mixture of both when the product comprises inulin and fructooligosaccharides.

In some cases, the hair was rinsed after application of the inulins and/or fructooligosaccharides after 5 minutes of immersion in the aqueous solution of active agents, whereas in other cases, the hair was not rinsed.

The tensiometer used is the tensiometer Krüss K100SF.

Sampling protocol

10 hairs per lock were measured.

For each hair, the following protocol is used:
The hair is cut from the lock using clean scissors without other handling, and handled with clean tweezers.
It is fixed on its upper portion to a sheet of paper by means of a double-sided adhesive.
For each measurement, a sample of a length of 15 mm is cut using clean scissors.
The first 3 samples are used to measure the wetted length.
The next 3 samples are used to measure the contact angle.
The sample is fixed on the adhesive surface of the sample holder over a length of approximately 5 mm (direct attachment to the sample holder without other handling). The sample must be as straight as possible, to come into contact perpendicularly with the liquid so as not to distort the measurement.

The measurement is carried out 5 mm from the bottom of the sample.

### Measurement Conditions

Measurement of wetted length

| Parameters: | Value |
|---|---|
| Rate of detection | 6 mm/min |
| Detection sensitivity | 5.10⁻⁵ g |
| Measurement of the speed | 3 mm/min |
| Difference in position | 0.2 mm |
| Depth of immersion | 5 mm |

### Testing of the Water

The measurements are carried out with tap water which is systematically tested every day during the measurements.

### Results:

| temperature | pH | Surface tension (mN/m) |
|---|---|---|
| 23° C. | 7.4 ± 1 | 72.7 mN/m (s = 0.1) |

### Results of the Hydrophobicity Tests:

The results are given by means of a calculation of a Mann-Whitney W test to compare the medians of the two samples compared, the samples being independent. The samples compared are hair damaged by bleaching (D2) and hair damaged by bleaching (D2) and treated with the aqueous solution of active agents.
Results of Hydrophobicity Tests 1% (Active gent A: Inulin-Metlin®, active agent B: Metlos®)

| | References | | | |
|---|---|---|---|---|
| | Lock 1: natural hair | | Lock 2: damaged (bleached) hair | |
| | Contact angle (°) | | Contact angle (°) | |
| | reg | mean | reg | mean |
| mean | 102.4 | 101.5 | 78.2 | 77.8 |
| Standard | 2.9 | 2.4 | 11.8 | 11.0 |
| deviation | | | | |
| Coeff. | 2.8 | 2.3 | 15.1 | 14.1 |
| variation % | | | | |

| | A/B ratio | | | | | | | | | |
|---|---|---|---|---|---|---|---|---|---|---|
| | 100/0 | | 75/25 | | 50/50 | | 25/75 | | 0/100 | |
| | Non-rinsed | | | | | | | | | |
| | Lock 3 | | Lock 4 | | Lock 5 | | Lock 6 | | Lock 7 | |
| | reg | reg | | mean | reg | mean | reg | mean | reg | mean |
| mean | 91.8 | 90.2 | 83.7 | 83.7 | 87.1 | 85.2 | 84.5 | 82.6 | 84.4 | 82.7 |
| Standard | 7.4 | 7.8 | 13.5 | 12.3 | 13.2 | 13.6 | 11.5 | 11.5 | 15.2 | 15.7 |
| deviation | | | | | | | | | | |
| Coeff. | 8.1 | 8.7 | 16.1 | 14.7 | 15.2 | 15.9 | 13.6 | 13.9 | 18.1 | 19.0 |
| variation % | | | | | | | | | | |
| | A/B ratio | | | | | | | | | |
| | 100/0 | | 75/25 | | 50/50 | | 25/75 | | 0/100 | |
| | Rinsed | | | | | | | | | |
| | Lock 8 | | Lock 9 | | Lock 10 | | Lock 11 | | Lock 12 | |
| | reg | mean | reg | mean | reg | mean | reg | mean | reg | mean |
| mean | 90.8 | 90.5 | 87.7 | 85.7 | 87.7 | 84.7 | 83.4 | 82.1 | 87.4 | 85.9 |
| Standard | 11.6 | 11.4 | 7.0 | 7.4 | 10.6 | 11.4 | 9.5 | 8.8 | 8.3 | 8.7 |
| deviation | | | | | | | | | | |
| Coeff. | 12.7 | 12.6 | 8.0 | 8.6 | 12.1 | 13.4 | 11.4 | 10.8 | 9.5 | 10.1 |
| variation % | | | | | | | | | | |

A significant difference in relation to D2 is observed.

Results of Hydrophobicity Tests 0.5% and 0.25% (Active agent A: Inulin-Metlin, active agent B: Metlos®)

| | | | Lock 2: Damaged (bleached) | |
|---|---|---|---|---|
| | Lock 1: natural hair | | hair | |
| | Contact angle (°) | | Contact angle (°) | |
| References | reg | mean | reg | mean |
| mean | 105.6 | 104.2 | 68.0 | 67.2 |
| Standard | 3.4 | 3.5 | 15.3 | 16.6 |
| deviation | | | | |
| Coeff. | 3.2 | 3.4 | 22.5 | 24.6 |
| variation % | | | | |

A significant difference in relation to D2 is observed.

| | 50/50-0.5% | | 50/50-25% | |
|---|---|---|---|---|
| | Contact angle (°) | | Contact angle (°) | |
| A/B ratio | reg | mean | reg | mean |
| mean | 77.6 | 76.8 | 69.5 | 68.0 |
| Standard deviation | 13.2 | 12.2 | 7.1 | 6.5 |
| Coeff. variation % | 17.0 | 15.8 | 10.2 | 9.6 |

P = 0.00355455**s
P = 0.0929457****ns

A significant difference in relation to D2 is observed for the 50/50 ratio at 0.5%.

Thus, a significant difference for the rinsed 50/50 ratio at 0.5% compared to the bleached control is observed, and a non-significant difference compared to the rinsed 50/50 at 1%.
Results of Hydrophobicity Tests All ratios rinsed and not rinsed at 1% +rinsed 50/50 tests at 0.5% and 0.25%.

A significant provision of hydrophobicity is observed for the treatments:
Rinsed at 1%: all the ratios (less for 25/75)
Rinsed at 0.5% for the 50/50 ratio
Not rinsed at 1% for all the ratios (less for 75/25 and 0/100)

### Example 3: Formulation of an Agave Extract in a Concentrated Serum for Plant-Based Hair Care

| | Percentage (% |
|---|---|
| Components (INCI name) | by weight) |
| AQUA | 55.575 |
| GELLAN GUM | 0.150 |
| SODIUM CHLORIDE | 0.075 |
| SODIUM BENZOATE | 0.500 |
| POTASSIUM SORBATE | 0.200 |
| SALICYLIC ACID | 0.150 |
| BETAINE | 2.000 |
| LACTIC ACID & AQUA | 0.150 |
| PANTHENOL | 0.200 |
| ALOE BARBADENSIS LEAF JUICE | 40.000 |
| INULIN | 1.000 |

### Example 4: Formulation of an Agave Extract in a Concentrated Serum for Plant-Based Hair Care

| | Percentage (% |
|---|---|
| Components (INCI name) | by weight) |
| AQUA | 54.575 |
| GELLAN GUM | 0.150 |
| SODIUM CHLORIDE | 0.075 |
| SODIUM BENZOATE | 0.500 |
| POTASSIUM SORBATE | 0.200 |
| SALICYLIC ACID | 0.150 |
| BETAINE | 2.000 |
| LACTIC ACID & AQUA | 0.150 |
| PANTHENOL | 0.200 |
| ALOE BARBADENSIS LEAF JUICE | 40.000 |
| FRUCTOOLIGOSACCHARIDES | 1.000 |
| INULIN | 1.000 |

### Example 5: Formulation of an Agave Extract in a Shampoo

| | Percentage (% |
|---|---|
| Components (INCI name) | by weight) |
| AQUA | 76.660 |
| GUAR HYDROXYPROPYLTRIMONIUM CHLORIDE | 0.150 |
| CITRIC ACID | 0.090 |
| SODIUM BENZOATE | 0.500 |
| ZINC GLUCONATE | 0.500 |
| SALICYLIC ACID | 0.050 |
| FRUCTOOLIGOSACCHARIDES | 0.125 |
| INULIN | 0.125 |
| COCO-GLUCOSIDE & GLYCERYL OLEATE & AQUA | 3.000 |
| DECYL GLUCOSIDE & AQUA | 3.000 |
| AMMONIUM LAURYL SULFATE & AQUA | 10.400 |
| COCAMIDOPROPYL BETAINE & AQUA | 4.000 |
| PARFUM | 0.400 |
| URTICA DIOICA EXTRACT & AQUA & GLYCERIN | 1.000 |

### Example 6: Formulation of an Agave Extract in a Nutritive Hair Care in the Form of Hair Conditioner

| | Percentage (% |
|---|---|
| Components (INCI name) | by weight) |
| AQUA | 87.967 |
| INULIN | 0.250 |
| FRUCTOOLIGOSACCHARIDES | 0.250 |
| PANTHENOL | 0.100 |
| SODIUM BENZOATE | 0.350 |
| BEHENTRIMONIUM CHLORIDE & ISOPROPYL | 3.000 |
| ALCOHOL | |
| STEARYL ALCOHOL | 3.500 |
| CETYL ALCOHOL | 2.000 |
| PERSEA GRATISSIMA OIL | 1.000 |
| COCOS NUCIFERA OIL | 1.000 |
| PARFUM | 0.400 |
| OLEA EUROPAEA FRUIT OIL & BETA-CAROTENE | 0.003 |
| CITRIC ACID | 0.180 |

### REFERENCE LIST

### 1. WO200790554.

2. María de la Soledad ALONSO GUTIÉRREZ, thesis, “Valorisation de la bagasse de l'agave tequilana W. cv azul : caractérisation, étude de la digestibilité et de la fermentation des sucres” [Utilizing bagasse of Agave tequilana W cv azul: characterization, study of digestibility and of sugar fermentation], 2005.
3. López, M.G. ; Mancilla, N.A. ; Mendoza-Díaz, G. (2003). : Molecular structures of fructans from Agave tequilana Weber var. azul. J. Agric. Chem. 51, 7835-7840.
4. Prevention of hair surface aging, J. Cosmet. Sci., 62, 237-249 (March/April 2011), ERIK SCHULZE ZUR WIESCHE, ANDREA KÖRNER, KAROLA SCHÄFER, and FRANZ-JOSEF WORTMANN, Henkel AG & Co. KGaA, Hohenzollemring 127-129, 22763 Hamburg, Germany (E.S.z.W.), DWI an der RWTH Aachen e.V., Pauwelsstr. 8, 52056 Aachen, Germany (A.K., K.S.), and School of Materials, The University of Manchester, Manchester M13 9PL, UK (F-J.W.).

1. The non-therapeutic cosmetic use of fructans of Agave tequilana comprising natural inulins for protecting the hair fiber by improving the hydrophobicity of the hair fiber.

2. The use as claimed in claim 1, wherein said fructans have a degree of polymerization of 3 to 29.

3. The use as claimed in claim 1, wherein said fructans are inulins having a degree of polymerization greater than 10.

4. The use as claimed in claim 3, wherein said inulins have a degree of polymerization of 10 to 60.

5. The use as claimed in claim 1, wherein said fructans further comprise fructooligosaccharides having a degree of polymerization less than or equal to 10.

6. The use as claimed in claim 1, wherein the ratio of inulins to fructooligosaccharides is from 100:0 to 0:100, the limit value of 0:100 not being included.

7. The use as claimed in claim 6, wherein the ratio of inulins to fructooligosaccharides is equal to 50:50.

8. The use as claimed in claim 1, wherein said fructans are present in a cosmetic composition comprising a cosmetically acceptable support.

9. The use as claimed in claim 8, wherein said cosmetically acceptable support is chosen from the group comprising shampoos, salves, lotions, creams, oils, mousses, milks, ointments, powders, solutions, gels, balms, butters, suspensions, soaps, sprays, emulsions and serums.

10. A non-therapeutic cosmetic process comprising the therapeutic application of fructans of Agave tequilana comprising inulins having a degree of polymerization greater than 10, for protecting the hair fiber by improving the hydrophobicity of the hair fiber, said application being optionally followed by rinsing.

11. The process as claimed in claim 10, wherein said application is not followed by rinsing.

12. The process as claimed in claim 10, on natural hair, damaged hair, dry hair, dyed hair or brittle hair.
